(19) United States
(12) Patent Application Publication
(10) Pub. No.: US 2025/0347601 A1
(43) Pub. Date: Nov. 13, 2025

(54) METHOD AND SYSTEM FOR EVALUATING THE PERFORMANCE OF METALLIC MATERIALS BASED ON CREEP-FATIGUE INTERACTION

(71) Applicant: Tianjin University, Tianjin City (CN)

(72) Inventors: Lianyong Xu, Tianjin City (CN); Lei Zhao, Tianjin City (CN); Yongdian Han, Tianjin City (CN); Chengchong Hu, Tianjin City (CN); Kangda Hao, Tianjin City (CN)

(21) Appl. No.: 19/191,465

(22) Filed: Apr. 28, 2025

(30) Foreign Application Priority Data
May 9, 2024 (CN) 202410566333.4

(51) Int. Cl. G01N 3/08; G01N 33/2045

(52) U.S. Cl. CPC G01N 3/08; G01N 33/2045; G01N 2203/0071; G01N 2203/0073

(57) ABSTRACT

A method and system for evaluating the performance of metallic materials based on creep-fatigue interaction, comprising: based on elastic-plastic property data and creep property data of metallic materials, calculating the monotonic crack tip opening displacement and cyclic crack tip opening displacement caused by plasticity or creep of short cracks under maximum far-field stress and far-field cyclic stress, respectively; using the linear superposition method to obtain the total monotonic crack tip opening displacement and cyclic crack tip opening displacement of short cracks under the combined creep-fatigue action; by calculating the short crack growth rate of the metallic material under creep-fatigue interaction, the performance of the metallic material is evaluated. The invention solves the problems of traditional methods, such as relying on a large amount of crack growth experimental data and insufficient consideration of creep-fatigue short crack effects.

### 1. TECHNICAL FIELD

The invention relates to the technical field of metallic material performance evaluation, in particular to a method and system for evaluating the performance of metallic materials based on creep-fatigue interaction.

### 2. BACKGROUND ART

Creep-fatigue loads are generated in energy equipment during operation due to start-up/shutdown processes and power adjustments. Different from single creep or fatigue loads, under the creep-fatigue interaction, structural components may fail at an accelerated rate, posing a threat to the stable operation of structural parts in energy units. The calculation of creep-fatigue crack growth rate is of vital significance in assessing the structural integrity of high-temperature components in modern energy equipment.

Traditional methods for calculating creep-fatigue crack growth rates require numerous creep-fatigue crack growth tests to determine the corresponding material parameters. Meanwhile, due to the short crack effect, traditional fracture mechanics parameters cannot be used to calculate the growth rate of creep-fatigue short cracks. Extensive research indicates that the short crack growth stage accounts for over 70% of the material' s crack growth life. Therefore, there is an urgent need to develop a performance evaluation technique for metallic materials based on creep-fatigue interaction. This technique would evaluate the creep-fatigue performance of metallic materials simply and efficiently by calculating short cracks in metallic materials under creep-fatigue interaction.

### 3. SUMMARY OF THE INVENTION

In order to address the above problem, the invention provides a technique for evaluating the performance of metallic materials based on creep-fatigue interaction. Based on the evolution laws of the plastic zone and creep zone at the tip of short cracks under creep-fatigue loading, by calculating the damage induced by creep and fatigue loading through a dislocation model, a short crack growth rate model for metallic materials under creep-fatigue interaction is established. All experimental data for this method are derived from straightforward uniaxial test results, thereby addressing problems such as the existing methods' reliance on extensive crack growth test data and incomplete consideration of creep-fatigue short crack effects. This provides a theoretical foundation for structural integrity assessment of high-temperature components in energy equipment operating under complex working conditions.

In order to realize the above objects, the invention provides the following technical scheme: a method for evaluating the performance of metallic materials based on creep-fatigue interaction, comprising the following steps:
based on elastic-plastic property data of metallic materials and in combination with evolution law of the plastic zone at crack tip, obtaining a first monotonic crack tip opening displacement caused by plasticity under maximum far-field stress acting on short cracks, as well as a first cyclic crack tip opening displacement caused by plasticity under far-field cyclic stress;
based on creep property data of metallic materials and in combination with the evolution law of the creep zone at crack tip, using a dislocation model to obtain a second monotonic crack tip opening displacement caused by creep under the maximum far-field stress acting on short cracks, as well as a second cyclic crack tip opening displacement caused by creep under the far-field cyclic stress;
based on the first monotonic crack tip opening displacement, the first cyclic crack tip opening displacement, the second monotonic crack tip opening displacement, and the second cyclic crack tip opening displacement, obtaining a total monotonic crack tip opening displacement and cyclic crack tip opening displacement of short cracks under a combined creep-fatigue action; by calculating a short crack growth rate of metallic materials under the creep-fatigue interaction, evaluating the performance of metallic materials;

Preferably, in the process of obtaining the first monotonic crack tip opening displacement, the first monotonic crack tip opening displacement is obtained based on the yield strength, elastic modulus, Poisson' s ratio, far-field maximum stress, and short crack length of the metallic material.

Preferably, in the process of obtaining the first cyclic crack tip opening displacement, the first cyclic crack tip opening displacement is generated by obtaining the load ratio, based on the yield strength, elastic modulus, Poisson' s ratio, far-field maximum stress, and short crack length of the metallic material.

Preferably, in the process of obtaining the second monotonic crack tip opening displacement, the second monotonic crack tip opening displacement is obtained by obtaining the equivalent creep yield strength of the metallic material, based on the yield strength, elastic modulus, Poisson' s ratio, far-field maximum stress, and short crack length of the metallic material.

Preferably, in the process of obtaining the second cyclic crack tip opening displacement, the second cyclic crack tip opening displacement is generated by obtaining the load ratio and the equivalent creep yield strength, based on the yield strength, elastic modulus, Poisson' s ratio, far-field maximum stress, and short crack length of the metallic material.

Preferably, in the process of obtaining the equivalent creep yield strength, the co-planar creep zone size related to creep time is obtained; based on the short crack length and the far-field maximum stress, the equivalent creep yield strength is obtained.

Preferably, in the process of obtaining the co-planar creep zone size, the stress intensity factor for Mode I cracks, the creep stress index of the metallic material, the specimen thickness, creep time, and the correction parameter related to the creep stress index are obtained; based on the elastic modulus of the metallic material, the co-planar creep zone size is obtained.

Preferably, in the process of obtaining the correction parameter, the correction parameter is obtained by acquiring material parameters related to the creep stress index, based on the creep stress index.

Preferably, in the process of obtaining the total monotonic crack tip opening displacement and cyclic crack tip opening displacement of short cracks under the combined creep and fatigue action, the total monotonic crack tip opening displacement and cyclic crack tip opening displacement of short cracks under the combined creep and fatigue action are obtained by using the linear superposition method.

The invention also provides a system for evaluating the performance of metallic materials based on creep-fatigue interaction, comprising:
a first data calculation module: based on elastic-plastic property data of metallic materials and in combination with the evolution law of the plastic zone at crack tip, obtaining the first monotonic crack tip opening displacement caused by plasticity under maximum far-field stress acting on short cracks, as well as the first cyclic crack tip opening displacement caused by plasticity under far-field cyclic stress;
a second data calculation module: based on creep property data of metallic materials and in combination with the evolution law of the creep zone at crack tip, using the dislocation model to obtain the second monotonic crack tip opening displacement caused by creep under the maximum far-field stress acting on short cracks, as well as the second cyclic crack tip opening displacement caused by creep under the far-field cyclic stress;
an evaluation module: based on the first monotonic crack tip opening displacement, the first cyclic crack tip opening displacement, the second monotonic crack tip opening displacement, and the second cyclic crack tip opening displacement, obtaining the total monotonic crack tip opening displacement and cyclic crack tip opening displacement of short cracks under the combined creep-fatigue action; by calculating the short crack growth rate of metallic materials under the creep-fatigue interaction, evaluating the performance of metallic materials

The advantageous effects of the invention are as follows:
the invention takes into account the accelerating effect of creep-fatigue interaction on the growth rate of short cracks, addressing the problem that traditional methods cannot accurately calculate the creep-fatigue short crack growth rate;
the invention is a method for calculating the short crack growth rate based on uniaxial tension and creep test data, resolving the problem that traditional methods rely heavily on extensive crack growth test data;
the proposed method for calculating the short crack growth rate under creep-fatigue interaction in this invention can yield unified prediction results for the short crack growth rate at different creep time, overcoming the limitations of creep dwell time effects in traditional methods.

Verification has demonstrated that this invention performs well in calculating the growth rate of creep-fatigue short cracks.

### 4. BRIEF DESCRIPTION OF ACCOMPANYING DRAWINGS

In order to explain the technical schemes in the embodiments of the invention or prior art more clearly, the accompanying drawings needed to be used in the description of the embodiments or the prior art will be briefly introduced below. Obviously, the accompanying drawings in the following description are only are some embodiments of the invention. For those of ordinary skill in the art, other accompanying drawings can be obtained based on these accompanying drawings without exerting creative efforts.

FIG. 1 is a flowchart of the method of the invention;

FIG. 2 is a schematic diagram of the parameter fitting of uniaxial creep test data in the invention.

FIG. 3 is a schematic diagram of the parameter fitting of uniaxial tensile test data in the invention.

FIG. 4 is a graph of the creep-fatigue short crack growth rate calculation results in the invention.

FIG. 5 is a graph of the creep-fatigue short crack growth rate calculation results based on test data by using the traditional method in the invention.

### 5. SPECIFIC EMBODIMENT OF THE INVENTION

In order to make the objects, technical schemes and advantages of the embodiments of the invention clearer, the technical schemes in the embodiments of the invention will be clearly and completely described below in combination with the accompanying drawings in the embodiments of the invention, obviously, the described embodiments are some, but not all embodiments of the invention. The components of the embodiments of the invention generally described and illustrated in the drawings herein may be arranged and designed in a variety of different configurations. Therefore, the following detailed description of the embodiments of the invention provided in the accompanying drawings is not intended to limit the scope of the invention to be protected, but merely represents selected embodiments of the invention. Based on the embodiments of the invention, all other embodiments obtained by those skilled in the art without creative work are within the protection scope of the invention.

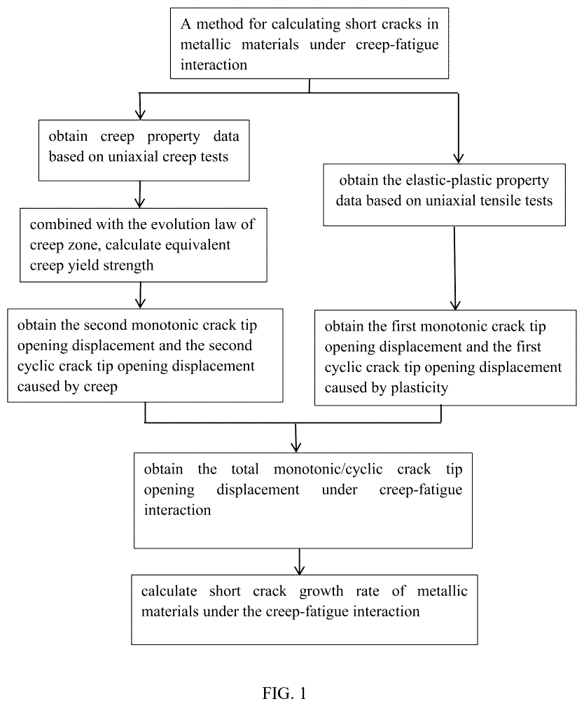

As shown in FIG. 1 to FIG. 5, In order to address the above problem, the invention provides a method and system for evaluating the performance of metallic materials based on creep-fatigue interaction; by calculating the short cracks of metallic materials under creep-fatigue interaction, the creep-fatigue performance of metallic materials is evaluated in a simple and efficient manner, including the following step: constructing a short crack growth rate calculation model under creep-fatigue interaction. The prediction model utilizes creep performance data based on uniaxial creep tests and elastic-plastic property data based on uniaxial tensile tests to calculate the creep-fatigue short crack growth rate of the material; by using elastic-plastic property data from uniaxial tensile tests, combined with the evolution law of the plastic zone at crack tip, and applying the dislocation model, the first monotonic crack tip opening displacement

$${δ}^{p} \text{?}$$

$$\text{?} \text{indicates text missing or illegible when filed}$$

caused by plasticity under maximum far-field stress acting on short cracks and the first cyclic crack tip opening displacement

$${δ}^{p} \text{?}$$

$$\text{?} \text{indicates text missing or illegible when filed}$$

caused by plasticity under far-field cyclic stress are obtained; by utilizing creep performance data based on uniaxial creep tests and combined with the evolution law of the creep zone at crack tip, and applying the dislocation model, the second monotonic crack tip opening displacement

$$δ \text{?}$$

$$\text{?} \text{indicates text missing or illegible when filed}$$

caused by creep under maximum far-field stress acting on short cracks and the second cyclic crack tip opening displacement

$$δ \text{?}$$

$$\text{?} \text{indicates text missing or illegible when filed}$$

caused by creep under far-field cyclic stress are obtained; based on the monotonic/cyclic crack tip opening displacement caused by creep/fatigue, the total monotonic crack tip opening displacement δₜ and cyclic crack tip opening displacement δᵣ of short cracks under the combined creep-fatigue action are calculated; based on the obtained δₜ and δᵣ, the creep-fatigue short crack growth rate of the material is calculated by using the crack growth rate model under the creep-fatigue interaction. The invention solves the problems of traditional methods, such as relying on a large amount of crack growth experimental data and insufficient consideration of creep-fatigue short crack effects.

The invention provides a method for calculating short cracks in metallic materials under creep-fatigue interaction, comprising the following steps:
step 1: constructing a short crack growth rate calculation model under the creep-fatigue interaction. This prediction model uses creep property data from uniaxial creep tests and elastic-plastic property data from uniaxial tensile tests to calculate the creep-fatigue short crack growth rate of the material;
step 2: by utilizing the elastic-plastic property data based on uniaxial tensile tests and combined with evolution law of the plastic zone at crack tip, and applying the dislocation model, the first monotonic crack tip opening displacement

$${δ}^{p} \text{?}$$

$$\text{?} \text{indicates text missing or illegible when filed}$$

caused by plasticity under maximum far-field stress acting on short cracks and the first cyclic crack tip opening displacement

$${δ}^{p} \text{?}$$

$$\text{?} \text{indicates text missing or illegible when filed}$$

caused by plasticity under far-field cyclic stress are obtained;
step 3: by utilizing creep performance data based on uniaxial creep tests and combined with the evolution law of the creep zone at crack tip, and applying the dislocation model, the second monotonic crack tip opening displacement

$$δ \text{?}$$

$$\text{?} \text{indicates text missing or illegible when filed}$$

caused by creep under maximum far-field stress acting on short cracks and the second cyclic crack tip opening displacement

$$δ \text{?}$$

$$\text{?} \text{indicates text missing or illegible when filed}$$

caused by creep under far-field cyclic stress are obtained;
step 4: based on the monotonic/cyclic crack tip opening displacement caused by creep/fatigue, the total monotonic crack tip opening displacement δₜ and cyclic crack tip opening displacement δᵣ of short cracks under the combined creep-fatigue action are calculated;
step 5: based on the obtained δₜ and δᵣ, the creep-fatigue short crack growth rate of the material is calculated by using the crack growth rate model under the creep-fatigue interaction.

When constructing the short crack growth rate calculation model under the creep-fatigue interaction in step 1, the crack growth rate is expressed as the crack growth per cycle: (dc)_{cycle}.

The formula used in constructing the short crack growth rate calculation model under the creep-fatigue interaction in step 1:

$${( d c )}_{cycle} = B ( {δ}_{t} {δ}_{r} ) a ;$$

wherein β and α are fitting parameters of the model.

In the process of obtaining the first monotonic crack tip opening displacement

$$δ \text{?}$$

$$\text{?} \text{indicates text missing or illegible when filed}$$

caused by plasticity under maximum far-field stress acting on short cracks in step 2, its expression is as follows:

$$δ \text{?} = \frac{8 c ( 1 - {v}^{2} ) {σ}_{y} ln [ sec ( \frac{π {σ}_{peak}}{2 {σ}_{y}} ) ]}{π E} ;$$

$$\text{?} \text{indicates text missing or illegible when filed}$$

wherein σ_{y} is the yield strength of the material, E is the elastic modulus of the material, v is the Poisso's ratio of the material, σₚₑₐₖ is the maximum far-field stress, and c is the short crack length.

In the process of obtaining the first cyclic crack tip opening displacement

$${δ}_{r}^{p}$$

caused by plasticity under far-field cyclic stress in step 2, its expression is as follows:

$$δ \text{?} = \frac{16 c ( 1 - {v}^{2} ) {σ}_{y} ln [ sec ( \frac{π {σ}_{peak} ( 1 - R )}{4 {σ}_{y}} ) ]}{π E} ;$$

$$\text{?} \text{indicates text missing or illegible when filed}$$

wherein R is the load ratio.

In the process of obtaining the second monotonic crack tip opening displacement

$${δ}_{t}^{c}$$

caused by creep under maximum far-field stress acting on short cracks in step 3, its expression is as follows:

$$δ \text{?} \frac{8 c ( 1 - {v}^{2} ) {σ}_{y} ln [ sec ( \frac{π {σ}_{peak}}{2 {σ}_{y}^{cr}} ) ]}{π E} ;$$

$$\text{?} \text{indicates text missing or illegible when filed}$$

Preferably, when obtaining the material' s equivalent creep yield strength, its expression is as follows:

$${σ}_{y}^{cr} = \frac{π {σ}_{peak}}{2 arccos ( \frac{c}{{ρ}^{cr} ( t ) + c} )} ;$$

wherein ρ^{cr}(t) is the co-planar creep zone size related to the creep time.

In the process of obtaining the co-planar creep zone size related to the creep time ρ^{cr}(t), its expression is as follows:

$${ρ}^{cr} ( t ) = \frac{1}{2 π} {( \frac{{K}_{I}}{E} )}^{2} {{ψ}_{cr} [ \frac{{{WtE}^{m} ( m + 1 )}^{2}}{2 {mr}_{m}^{m + 1}} ]}^{\frac{2}{m - 1}} ;$$

wherein K₁ is the stress intensity factor for Mode I cracks, m is the creep stress index of the material, W is the specimen thickness, t is the creep time, and rₘ and ψ_{cr} are correction parameters related to the creep stress index m.

When obtaining the correction parameter rₘ related to the creep stress index m, its expression is as follows:

$${r}_{m} = {( \frac{π}{{η}_{m}} \frac{m + 1}{m} )}^{\frac{1}{m + 1}} ;$$

wherein ηₘ is the material parameter related to the creep stress index m.

When obtaining the correction parameter rₘ related to the creep stress index m, its value is determined by using an interpolation method based on Table 1.

TABLE 1

| m | 3 | 5 | 9 | 13 |
|---|---|---|---|---|
| rₘ | 3.86 | 3.41 | 3.03 | 2.87 |

When obtaining the correction parameter ψ_{cr} related to the creep stress index m, its value is determined by using the interpolation method based on Table 2.

TABLE 2

| m | 3 | 5 | 13 |
|---|---|---|---|
| ψ_{cr} | 0.25 | 0.32 | 0.38 |

using the linear superposition method to obtain the total monotonic crack tip opening displacement δₜ and cyclic crack tip opening displacement δᵣ of short cracks under the combined creep-fatigue action in step 4, its expression is as follows:

$$\{ \begin{matrix}{δ}_{t} = {δ}_{t}^{p} + {δ}_{t}^{c} \\ {δ}_{r} = {δ}_{r}^{p} + {δ}_{r}^{c}\end{matrix} .$$

When predicting the creep-fatigue short crack growth rate for high-temperature components of energy equipment, the materials are heat-resistant martensitic steel and austenitic steel, with a service temperature range of 500-700° C.

Embodiment 1: please refer to FIG. 1, the method for calculating short cracks in metallic materials under creep-fatigue interaction provided by the invention comprises short crack growth rate calculation method under creep-fatigue interaction on creep performance data based on uniaxial creep tests and on the elastic-plastic property data based on uniaxial tensile tests.

In order to better illustrate the method of the invention for calculating short cracks in metallic materials under creep-fatigue interaction, a single-edge notch specimen will be used for validation. The material used for validation is 316H heat-resistant steel, and uniaxial creep, uniaxial tensile, and creep-fatigue short crack growth tests are conducted under conditions of 550° C. The predicted creep-fatigue short crack growth rate is tested by using a load control method, maintaining the peak load, with a trapezoidal waveform.

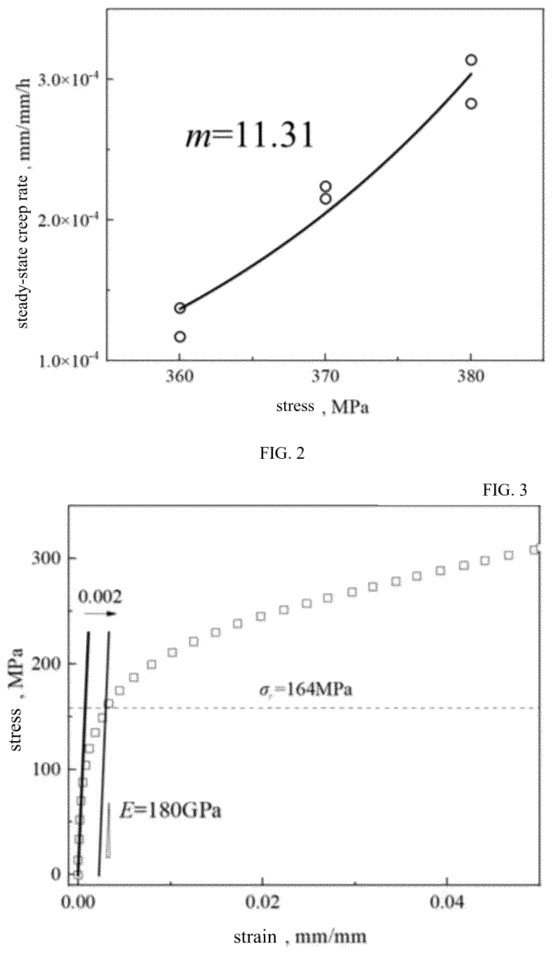

Step (1): based on the steady-state creep rate at different stress levels obtained from uniaxial creep tests, a nonlinear fitting method is used, as shown in FIG. 2, the creep stress index m under 550° C. conditions is 11.31. Further, the correction parameters rₘ and ψ_{cr} related to the creep stress index m are obtained as 1.01 and 0.3673, respectively.

Step (2): based on a tensile curve obtained from uniaxial tensile tests, as shown in FIG. 3, the elastic-plastic property under 550° C. conditions is obtained, including: the elastic modulus E of 180 GPa and the yield stress σ_{y} of 164 MPa, Additionally, the Poisson's ratio v for the metallic material is 0.3.

Step (3): based on the obtained elastic-plastic property, the obtaining the first monotonic crack tip opening displacement

$${δ}_{t}^{p}$$

caused by plasticity under maximum far-field stress acting on short cracks and the first cyclic crack tip opening displacement

$${δ}_{r}^{p}$$

caused by plasticity under far-field cyclic stress are obtained. Their expressions are as follows:

$${δ}_{t}^{p} = \frac{8 c ( 1 - {v}^{2} ) {σ}_{y} ln [ sec ( \frac{{πσ}_{peak}}{2 {σ}_{y}} ) ]}{π E} ; \\ {δ}_{r}^{p} = \frac{16 c ( 1 - {v}^{2} ) {σ}_{y} ln [ sec ( \frac{{πσ}_{peak} ( 1 - R )}{4 {σ}_{y}} ) ]}{π E} .$$

Step (4): based on the obtained creep property, the co-planar creep zone size ρ^{cr}(t) related to the creep time and the material' s equivalent creep yield strength are obtained. The expressions are as follows:

$${ρ}^{cr} ( t ) = \frac{1}{2 π} {( \frac{{K}_{I}}{E} )}^{2} {{ψ}_{cr} [ \frac{{{WtE}^{m} ( m + 1 )}^{2}}{2 {mr}_{m}^{m + 1}} ]}^{\frac{2}{m - 1}} ; \\ {σ}_{y}^{cr} = \frac{{πσ}_{peak}}{2 arccos ( \frac{c}{{ρ}^{cr} ( t ) + c} )} ;$$

Step (5): by using the results obtained in step (4); the second monotonic crack tip opening displacement

$${δ}_{t}^{c}$$

caused by creep under maximum far-field stress acting on short cracks and the second cyclic crack tip opening displacement

$${δ}_{r}^{c}$$

caused by creep under far-field cyclic stress are obtained, the expressions are as follows:

$${δ}_{t}^{c} = \frac{8 c ( 1 - {v}^{2} ) {σ}_{y}^{cr} ln [ sec ( \frac{{πσ}_{peak}}{2 {σ}_{y}^{cr}} ) ]}{π E} ; \\ {δ}_{r}^{c} = \frac{16 c ( 1 - {v}^{2} ) {σ}_{y}^{cr} ln [ sec ( \frac{{πσ}_{peak} ( 1 - R )}{4 {σ}_{y}^{cr}} ) ]}{π E} .$$

Step (6): by using the monotonic/cyclic crack tip opening displacement caused by creep/fatigue determined in step (4), the total monotonic crack tip opening displacement δₜ and cyclic crack tip opening displacement δᵣ of short cracks under the combined creep-fatigue action are calculated, its expression is as follows:

$$\{ \begin{matrix}{δ}_{t} = {δ}_{t}^{p} + {δ}_{t}^{c} \\ {δ}_{r} = {δ}_{r}^{p} + {δ}_{r}^{c}\end{matrix} .$$

Step (7): based on the obtained δₜ and δᵣ, the creep-fatigue short crack growth rate of the material is calculated by using the crack growth rate model under the creep-fatigue interaction, its expression is as follows

$${( dc )}_{c y c l e} = β ( {δ}_{t} {δ}_{r} ) α .$$

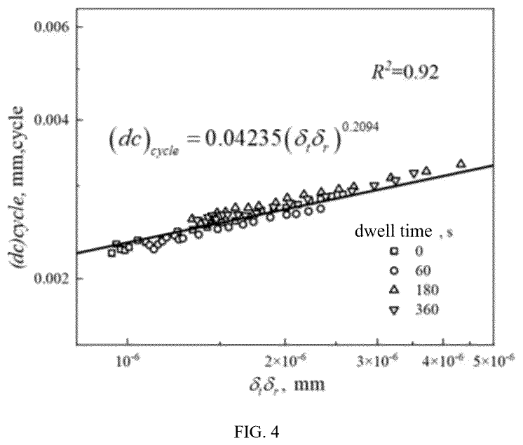

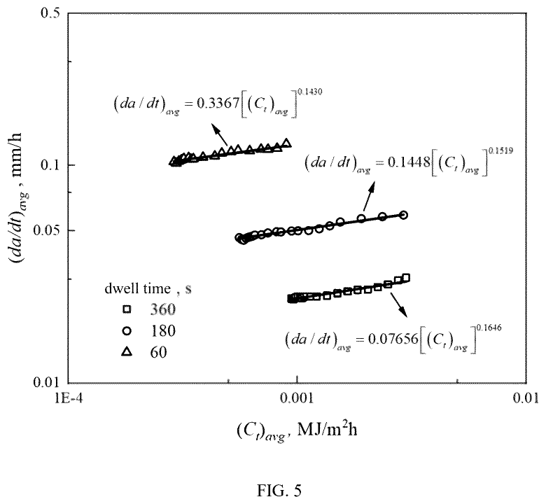

To verify the method for calculating short cracks in metallic materials under creep-fatigue interaction proposed in this invention, the creep-fatigue short crack growth rate under 550° C. conditions calculated by using this method is shown in FIG. 4. For comparison, the results of the traditional method for calculating short crack growth rate based on experimental data under creep-fatigue interaction are shown in FIG. 5. It can be seen that the method for calculating short cracks in metallic materials under creep-fatigue interaction in this invention provides good predictions of creep-fatigue short crack growth rates under different hold times, addressing the limitations of traditional methods, which cannot accurately describe short crack growth behavior and have dwell time-dependent prediction results.

The invention is described with reference to flowcharts and/or block diagrams of methods, devices (systems), and computer program products according to embodiments of the invention. It should be understood that each flow and/or block in the flowchart and/or block diagram, and a combination of flows and/or blocks in the flowchart and/or block diagram, can be implemented by computer program instructions. These computer program instructions can be provided to a processor of a general-purpose computer, a special-purpose computer, an embedded processor or other programmable data processing device to produce a machine, so that the instructions executed by the processor of the computer or other programmable data processing device produce a device for implementing the functions specified in one or more processes in the flowchart and/or one or more blocks in the block diagram.

In addition, the terms “first” and “second” are only used for descriptive purposes, and should not be understood as indicating or implying relative importance or implying the number of indicated technical features. Therefore, the features defined with “first” or “second” may expressly or implicitly include one or more of the features, and in the description of the invention, the meaning of “multiple” is two or more, unless otherwise expressly limited.

Obviously, those skilled in the art can make various changes and modifications to the invention without departing from the spirit and scope of the invention. Therefore, if these modifications and variations of the invention fall within the scope of the claims of the invention and their equivalents, the invention is also intended to include these modifications and variations.

1. (canceled)

2. (canceled)

3. (canceled)

4. A computer-implemented method for evaluating performance of metallic materials under creep-fatigue interaction, the method executed by a processor and comprising:
(a) obtaining elastic-plastic property data from a uniaxial tensile test of a metallic material specimen, including yield strength (σ_{y}), elastic modulus (E), Poisson's ratio (v);
(b) obtaining creep property data from a uniaxial creep test of the specimen, including a creep stress index (m);
(c) calculating, based on the elastic-plastic property data and evolution laws of a plastic zone at a crack tip:
(i) a first monotonic crack tip opening displacement
$$( {δ}_{t}^{c} )$$
caused by plasticity under maximum far-field stress (σₚₑₐₖ) acting on short cracks of length (c), using:
$${δ}_{r}^{p} = \frac{8 c ( 1 - {v}^{2} ) {σ}_{y} ln [ sec ( \frac{π {σ}_{peak}}{2 {σ}_{y}} ) ]}{π E}$$
(ii) a first cyclic crack tip opening displacement
$$( {δ}_{r}^{p} )$$
caused by plasticity under far-field cyclic stress, using:
$${δ}_{r}^{p} = \frac{16 c ( 1 - {v}^{2} ) {σ}_{y} ln [ sec ( \frac{π {σ}_{peak} ( 1 - R )}{4 {σ}_{y}} ) ]}{π E}$$
where R is a load ratio;
(d) calculating, based on the creep property data, evolution laws of a creep zone at the crack tip, and a dislocation model:
(i) a co-planar creep zone size (ρ^{cr}(t)) related to creep time (t), specimen thickness (W), and correction parameters (rₘ, ψ_{cr}) tied to the creep stress index (m), using:
$${ρ}^{cr} ( t ) = \frac{1}{2 π} {( \frac{{K}_{I}}{E} )}^{2} {{ψ}_{cr} [ \frac{{{WtE}^{m} ( m + 1 )}^{2}}{2 {mr}_{m}^{m + 1}} ]}^{\frac{2}{m - 1}}$$
wherein K_{I} is a Mode I stress intensity factor;
(ii) an equivalent creep yield strength
$$( {σ}_{y}^{cr} ) ,$$
using:
$${σ}_{y}^{cr} = \frac{π {σ}_{peak}}{2 arccos ( \frac{c}{{ρ}^{cr} ( t ) + c} )}$$
(iii) a second monotonic crack tip opening displacement
$$( {δ}_{t}^{c} )$$
caused by creep under ρₚₑₐₖ, using:
$${δ}_{t}^{c} = \frac{8 c ( 1 - {v}^{2} ) {σ}_{y}^{cr} ln [ sec ( \frac{π {σ}_{peak}}{2 {σ}_{y}^{cr}} ) ]}{π E}$$
(iv) a second cyclic crack tip opening displacement
$$( {δ}_{r}^{c} )$$
caused by creep under far-field cyclic stress, using:
$${δ}_{r}^{c} = \frac{16 c ( 1 - {v}^{2} ) {σ}_{y}^{cr} ln [ sec ( \frac{π {σ}_{peak} ( 1 - R )}{4 {σ}_{y}^{cr}} ) ]}{π E}$$
(e) applying linear superposition to calculate a total monotonic crack tip opening displacement (δₜ) and cyclic crack tip opening displacement (δᵣ) under combined creep-fatigue action:
$$\{ \begin{matrix}{δ}_{t} = {δ}_{t}^{p} + {δ}_{t}^{c} \\ {δ}_{r} = {δ}_{r}^{p} + {δ}_{r}^{c}\end{matrix}$$
(f) calculating a short crack growth rate ((dc)_{cycle}) to evaluate material performance, using:
$${( dc )}_{cycle} = β ( {δ}_{t} {δ}_{r} ) α .$$
where β and α are model fitting parameters derived from physical test data;
(g) outputting the short crack growth rate to a display or storage medium for structural integrity assessment of energy equipment components operating at 500-700° C.

5. The method of claim 4, wherein obtaining the correction parameter (rₘ) comprises:
retrieving a material parameter (ηₘ) associated with the creep stress index (m);
calculating rₘ using:
$${r}_{m} = {( \frac{π}{{η}_{m}} \frac{m + 1}{m} )}^{\frac{1}{m + 1}}$$
wherein ηₘ is determined via interpolation from empirical data mapping m to ηₘ.

6. The method of claim 4, wherein the correction parameters (rₘ, ψ_{cr}) are obtained by interpolating values from predefined tables based on the creep stress index (m):
rₘ values:
$$\begin{matrix}m = 3 \to {r}_{m} = 3.86 \\ m = 5 \to {r}_{m} = 3.41 \\ m = 9 \to {r}_{m} = 3.03 \\ m = 13 \to {r}_{m} = 2.87\end{matrix}$$
ψ_{cr} values:
$$\begin{matrix}m = 3 \to {ψ}_{cr} = 0.25 \\ m = 5 \to {ψ}_{cr} = 0.32 \\ m = 13 \to {ψ}_{cr} = 0.38 .\end{matrix}$$
